(19) United States
(12) Plant Patent Application Publication
(10) Pub. No.: US 2013/0333079 P1
(43) Pub. Date: Dec. 12, 2013

(54) Apple tree named 'MAC 2137'

(50) ;

(71) Applicant: David W. MacGregor, South Haven, MN (US)

(72) Inventor: David W. MacGregor, South Haven, MN (US)

(21) Appl. No.: 13/694,288

(22) Filed: Nov. 14, 2012

Related U.S. Application Data
(60) Provisional application No. 61/629,302, filed on Nov. 15, 2011.

(51) Int. Cl. A01H 5/00

(52) U.S. Cl. PLT/161

(57) ABSTRACT

A new and distinct apple tree named ‘MAC 2137’ is disclosed. The new apple is notable for its attractive appearance, superb texture, improved flavor and early ripening.

### CROSS-REFERENCE TO RELATED APPLICATIONS

Provisional application No. 61/629,302, filed on Nov. 15, 2011.

### STATEMENT REGARDING FEDERALLY SPONSORED RESEARCH OR DEVELOPMENT

None

### LATIN NAME OF THE GENUS AND SPECIES OF THE PLANT CLAIMED

Malus domestica Mill

### VARIETY DENOMINATION

‘MAC 2137’

### BRIEF DESCRIPTION OF THE PHOTOGRAPHS

FIG. 1 shows a ‘MAC 2137’ apple tree;

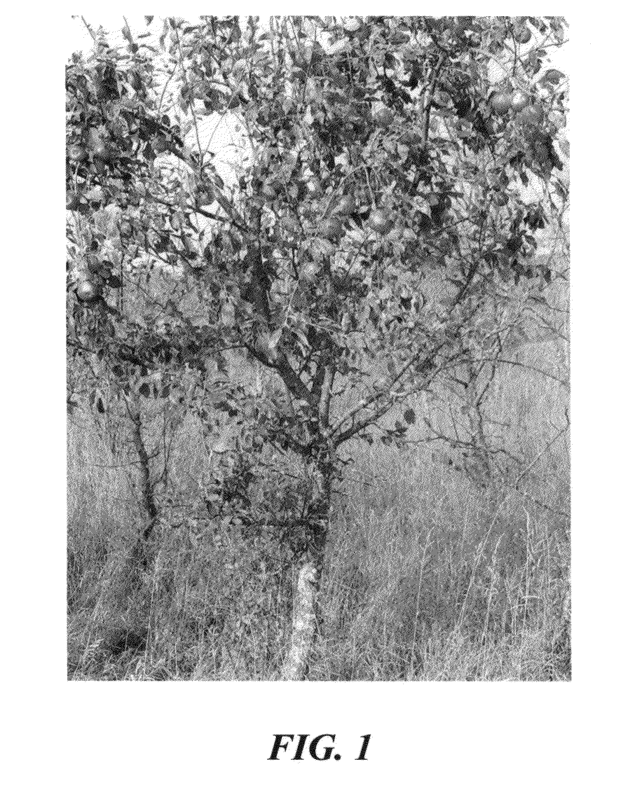

FIG. 2 shows the fruit of a ‘MAC 2137’ apple tree

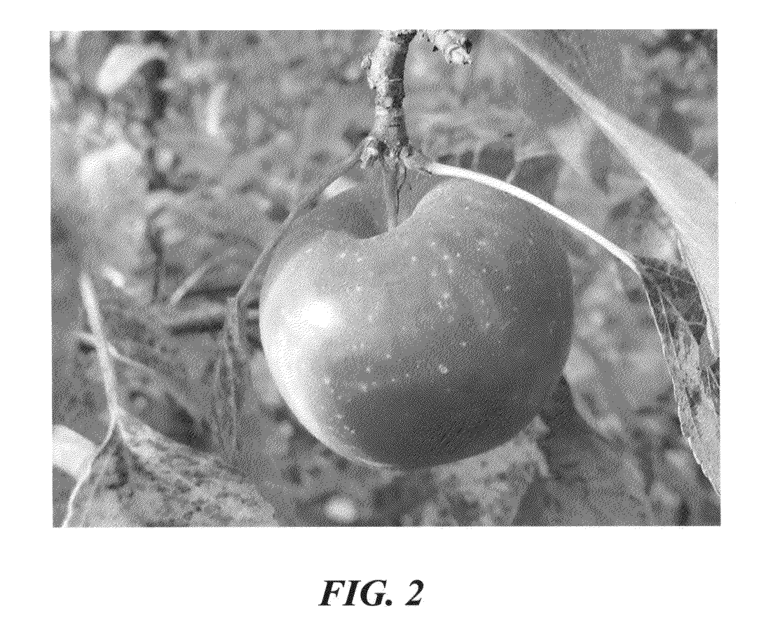

### BACKGROUND OF THE INVENTION

‘MAC 2137’ is a new and distinct apple tree. It was discovered by the inventor in 1998 as a seedling made from seeds saved from selected apples grown in a cultivated area in Fairhaven, Minn., and was found to be unlike other, known varieties. In 2003 scion wood of the parent tree was budded onto dwarfing rootstock, and second generation fruit was obtained. Further asexual propagation has been conducted, and the cultivar has been found to remain true to type through successive asexual propagations. The fruit of ‘MAC 2137’ is notable for its smaller size, earlier ripening, similar crunchy texture and sweeter flavor balance as compared to ‘Honeycrisp’ (U.S. Plant Pat. No. 7,197) apples.

### DETAILED BOTANICAL DESCRIPTION OF THE VARIETY

The following detailed botanical description is based on observations made during the 2012 growing season at Fairhaven, Minn. All colors are described according to The Royal Horticultural Society Colour Chart. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree: Type — Tall Spindle; Vigor — Strong prior to fruiting then Moderate; Height — 3 meters; Diameter — 1.3 meters; Trunk diameter at 20 cm above the graft union — 4.5 cm; Bark texture — smooth; Bark color grayed brown N199C and N199A; Lenticels — 1×2.5 mm, color — white N999D.
Branch: Length — 0.6 to 1 meter; Diameter — 1.5 cm; Crotch angle — 15 degrees; Color — grayed brown N199C; Lenticels — round to elongated, 1×1 mm or 1×2 mm, grayed brown N199D, 4 per cm2.
One year old shoot: Length — 0.2 to 0.6 meters; Color — Grayed purple N187A; Pubescence — Common, light to heavy, obscuring bark, underneath and toward terminal end; Color — Grayed brown N166A; Thickness — 3-4 mm at 10 cm from base; Internode length — 2-3.5 cm; Lenticels — 1 mm round to elongated, 7 per cm²
Winter hardiness: No injury in cold zone 4
Leaves: Shape — Ovate; Apex acuminate; Base equilateral; Length 7.5 cm-10 cm; Width — 3.5 cm-5.5 cm; Length/Width ratio — 1.89; Margin — serrate; Color of top surface — moderate olive green 137A; Color of lower surface moderate yellowish green 137C; Attitude in relation to shoot — Spirals around; Petiole — Length — 1.5 cm-3.5 cm; Diameter — 2 mm; Color — moderate yellowish green 146C
Flower: Bud (Dormant) — Quantity per spur — 1 to 2; length — 6 mm, diameter — 3.5 mm; color — 97C and 166A; Color (balloon stage) greyed brown N199D and strong purplish red 63B; Petals — Quantity per flower — 5; Length — 1.5 cm-2 cm; Width 1 cm-1.5 cm; Color of upper surface — white N999D with deep purplish pink 70C towards the margins; Color of lower surface — white N999D with strong purplish pink 55B shading toward margin or center; Arrangement — intermediate, touching; Diameter of open flower — 3.5 cm-4 cm; Pedicel — Length — 1.2 cm-2.3 cm; Diameter — 1.4 mm-2 mm; Color — Top — strong yellowish green 145A, Underneath — brownish orange 166C; Sepals — Quantity — 5; Color — pale yellowish green 149D; Size — 8 mm; Pistils — Size — 0.5 cm-1 cm; Color yellowish white 155D; Anthers — Quantity — 15-18; Size — 1.5 mm; Pollen — Color — vivid yellow 12A; Stigma — Size 1.4 cm; Color — yellowish green 150B; Style — Size 0.5 mm-1 mm; Color — brilliant yellowish green 150B; Ovary — Size — 4 mm; Color — moderate yellowish green 139C and purple N77B; Bloom — Date of first bloom — April 30^{th in} Fairhaven, Minn.; Date of full bloom — May 5^{th} in Fairhaven, Minn.
Fruit: Quantity per cluster — 1-3; Size — Height 4.7 cm, Width 5.7 cm; Weight — 3 oz; Ratio of Height to Width — 0.82; Shape — Oblate; Ribbing — insignificant; Depth of eye basin — 9.5 mm; Width of eye basin — 19 mm; Length of stalk — 1.5 cm-2.5 cm; Thickness of stalk — 1.5 mm; Width of stalk cavity — 12 mm; Depth of stalk cavity — 9.5 mm; Size of lenticels — 1 mm; Bloom of skin — pronounced; Greasiness of skin — Some when more than three weeks past prime harvest date; Background color of skin — 8C; Overcolor of skin — vivid red 45A; Amount of over color — 70% on exposed fruit; Intensity of overcolor — Strong; Pattern of over color — irregular blush; Flesh texture — extremely crisp; Aroma — mild apple; Juiciness — Juicy; Brix — 14°; Flesh coloration — yellowish white 155D; Stem coloration — light yellowish brown199C
Seeds: Quantity per fruit — 8-9; Shape — teardrop; Color — moderate brown 165A
Harvest: August 28^{th} — September 20^{th} in Fairhaven, Minn.
Use: Fresh Market.

1. We claim a new and distinct apple tree substantially as shown and described herein.
